(19) United States
(12) Patent Application Publication
(10) Pub. No.: US 2018/0028250 A1
(43) Pub. Date: Feb. 1, 2018

(54) METHOD & APPARATUS TO PERFORM CRYOTHERAPY

(71) Applicant: CSA Medical, Inc., Lexington, MA (US)

(72) Inventor: John P. O'Connor, Andover, MA (US)

(21) Appl. No.: 15/659,199

(22) Filed: Jul. 25, 2017

Related U.S. Application Data
(60) Provisional application No. 62/366,809, filed on Jul. 26, 2016.

(51) Int. Cl. A61B 18/02; A61F 2/958; A61H 39/06

(52) U.S. Cl. CPC A61B 18/02; A61H 39/06; A61F 2/958; A61B 2018/0231; A61B 2018/0212

(57) ABSTRACT

Cryosurgery systems, delivery apparatus and methods that provide for the application of cryogen to a treatment area via a low-profile, low pressure, closed-tipped probe. Cryogen is circulated through the probe, and vented to outside of the body, optionally under vacuum pressure, which contributes to increased cryogen throughput.

### PRIORITY

This application claims the benefit of priority under 35 U.S.C. §119 to U.S. provisional patent application Ser. No. 62/366,809, filed Jul. 26, 2016, which is incorporated by reference in its entirety and for all purposes.

### TECHNICAL FIELD

The present invention relates generally to cryosurgery apparatuses, systems and methods of treatment, and more particularly to improved cryogenic delivery to a treatment area via a low-profile, low pressure, closed-tipped catheter, needle or probe.

### BACKGROUND

The present invention relates to methods and devices for cryogenic treatment of organic tissue. Tissue ablation refers to the removal or destruction of tissue, or of tissue functions. Traditionally, invasive and non-invasive surgical procedures are used to perform tissue ablation. These surgical procedures required the cutting and/or destruction of tissue positioned between the exterior of the body and the site where the ablation treatment is conducted, referred to as the treatment area. Cryo ablation is an alternative in which tissue ablation is conducted by freezing diseased, damaged or otherwise unwanted target tissue. Appropriate target tissue may include, for example, cancerous or precancerous lesions, tumors (malignant or benign), damaged epithelium, fibroses and any other healthy or diseased tissue for which cryo ablation is desired.

As used typically, cryogen refers to any fluid (e.g., gas, liquefied gas or other fluid known to one of ordinary skill in the art) that has a sufficiently low boiling point to allow for therapeutically effective cryotherapy and is otherwise suitable for cryogenic surgical procedures. For example, acceptable fluids may have a boiling point below approximately negative (−) 150° C. The cryogen may be liquefied nitrogen, as it is readily available. Other fluids such as argon and air may also be used. Additionally, liquid helium, liquid oxygen, liquid nitrous oxide and other cryogens can also be used.

During operation of a cryosurgery system, a clinician, physician, surgeon, technician, or other operator delivers cryogen to the target tissue at the treatment are. The application of cryogen causes the target tissue to freeze or “cryofrost.” The physician may target the cryogen delivery visually utilizing laparoscopy, endoscopy, bronchoscopy, pleuroscopy, or other video assisted device or scope. The temperature range can be from 0° C. to negative (−) 195° C. This latter temperature in particular is the case of liquid nitrogen at low pressure.

Cryo ablation may be performed by using a system that sprays low-pressure cryogen directly onto target tissue or sprays cryogen within a balloon that is in contact with target tissue. Alternatively, cryogen is applied at high pressure from within the interior of a needle or probe, and the effect of the cryogen is realized by contact of the tip to or within the target tissue.

The advantage of direct spray or balloon catheters is the ability to deliver cryogen at low pressure, but extended treatment times may be required due to lower relative throughput of liquid nitrogen and the need to achieve near liquid nitrogen temperatures for treatment purposes. Converted gaseous nitrogen delivered within the body, in the case of direct spray, must be carried out of the body and released to the atmosphere typically by passive or active (under low suction) venting through an exhaust lumen or separate tubing. Attention to proper venting is necessary to avoid potentially harmful distention and pressure within the body if gaseous nitrogen accumulates. Circulation of gaseous nitrogen through a balloon catheter, must be done with attention to how the venting affects the dynamics of balloon expansion and deflation.

Existing cryotherapy needles or probes utilize the Joule-Thomson effect (primarily using argon gas) to generate a cold region near the tip of the needle. Such probes and needles, with closed-tip configurations and materials, in order to attain cryogenic treatment temperatures, use high input pressures up to 100 psi for liquid nitrogen or up to over 1,000 psi for Argon. The high pressure may increase throughput compared to low pressure systems, but such high pressures carry inherent dangers and typically require the probe systems to have larger profile needles.

There is, therefore, an existing need addressed by the present invention for cryosurgery apparatuses, systems and methods of treatment, that increase cryogen throughput, maintain low inlet flow pressure, and allows for reduced tip profile dimensions while achieving cryogen treatment temperatures at the target tissue with reduced treatment cycles.

### SUMMARY

The present invention in its various embodiments includes cryogenic delivery apparatuses, system and treatment methods. Converted cryogen, such as nitrogen gas, rather than being released within the body and either passively or actively vented from there, is circulated through a closed-tip catheter, needle or probe, and vented to outside of the body, optionally under vacuum pressure. A closed-tip configuration allows contact treatment of desired tissue regions with low-pressure input of a cryogen such as liquid nitrogen through lower profile devices, while maintaining or increasing throughput of liquid nitrogen and achieving liquid nitrogen temperatures at a more efficient rate.

In one aspect of the present invention, there is provided an advanced cryosurgery system that may include a console with on-board controls, a cryogen source, a vacuum source, and a delivery apparatus, among other components. The system may provide improved cryogen flow, flow control, suction, pressure sensing and temperature sensing, among other features.

In a further aspect, the system in various embodiments may include a temperature feedback loop with electronics to control cryogen delivery time with temperature reported. A thermocouple wire or other temperature sensor may be configured at or near the distal tip of a needle head to report temperatures used by the system in a feedback loop mode to control the cryogen dose.

In another aspect, various embodiments of tip designs and shaft configurations and dimensions for a delivery apparatus in accordance with the present invention, are contemplated. The catheter construction may include materials selected to maximize heat conductivity that allow for cryo cooling of a catheter fluid path ahead of a dual phase flow which may be achieved, for example, with a balance of metal or polymeric tubing and polymeric layering with metal braiding/coiling and a selection of diameters and lengths along the delivery shaft to deliver a desired cryogen flow rate.

In accordance with an aspect, various embodiments of the delivery apparatus may include one or more of: a proximal interface “bayonet” that can be connected to a console; an insulating sheath distributed over a proximal portion of a shaft of the delivery apparatus; a larger diameter proximal tube; an outer covering in the form of a polymeric layer to cover a portion or the entire length of the proximal tube to provide a fluid tight lumen; a smaller diameter distal tube of polymer and metal braid construction; a proximal or distal tube made of metal hypotube, with up to 100″ working length, with a varying laser cut profile; a polymeric shaft construction; and catheter markings or bands on a distal end to provide visual indication of the position and orientation of the tip.

In a further aspect of the present invention, in any of the various embodiments, a vacuum source may be included with the system or an outlet of the delivery apparatus is configured to accept a vacuum source. The vacuum may be controlled from a console of the system, and may be operated manually or automatically in connection with a feedback loop control to increase throughput of cryogen in the delivery apparatus and improve the overall efficiency of the systems and methods with respect to desired treatment goals and protocols.

Additionally, or alternatively, to the above or below, in yet another aspect, a cryosurgical system comprises a cryogen source, a vacuum source and a cryogen delivery apparatus. The delivery apparatus is configured to (i) connect to the vacuum source and the cryogen source, (ii) deliver cryogen in liquid form from the cryogen source through the apparatus at a low positive pressure to a treatment area, and (iii) remove cryogen in gaseous form from the treatment area through the apparatus at a negative pressure produced by the vacuum source. The cryogen delivery apparatus may be a catheter; the catheter may have a closed distal end. The closed distal end may have one or more blunt tips to contact a surface of a treatment area or may have one or more needle tips to penetrate a surface of the treatment area. The system may have a low positive pressure up to positive 20 psi. The system may have a negative pressure up to negative 15 psi. The cryogen source of the system may be nitrogen in liquid form. The system may further comprise a console having on-board controls and a temperature sensor in electrical communication with the controls. The controls and temperature sensor may be coupled to a closed distal end of a catheter in a feedback loop arrangement. The feedback arrangement may allow for control of a rate of cryogen delivered and removed by the system based on temperature measured by the sensor.

Additionally, or alternatively, to the above or below, in yet another aspect, an apparatus for delivery of cryogen to a treatment area within a body may include a proximal attachment end for connection to a cryogen source, and a closed distal end having a head with one or more low profile tips to contact the treatment area. The apparatus may include a shaft that may have a first inlet lumen and a second outlet lumen. The first inlet lumen may extend from the proximal end to deliver cryogen in liquid form to the one or more low profile tips under low positive pressure. The second outlet lumen may extend from the one or more low profile tips to vent cryogen in gaseous form from the treatment area to atmosphere outside the body. The apparatus may be a catheter with a proximal end for connecting to a cryogen source. The one or more low profile tips may have a blunt face to contact a surface of the treatment area, or the one or more low profile tips may be sufficiently sharp to penetrate a surface of the treatment area. An outer diameter of the first inlet lumen within the one or more low profile tips may be no more than 26 gauge. An outer diameter of the second outlet lumen within the one or more low profile tips may be no more than 19 gauge. The cryogen source may be nitrogen in liquid form. The low positive pressure for delivery of cryogen in liquid form may be up to positive 30 psi. The second outlet lumen may have a connection for a vacuum source. The vacuum source may be configured to vent cryogen in gaseous form. The vacuum pressure may be up to negative 15 psi. The first inlet lumen may be arranged co-axially within the second outlet lumen leaving a channel therebetween in fluid communication with the first inlet lumen. The channel may define a flow path to vent cryogen in gaseous form from the treatment area.

Additionally, or alternatively, to the above or below, in yet another aspect, a method to deliver cryotherapy to a treatment area comprises positioning a closed distal end of a cryoprobe in contact with the treatment area, delivering nitrogen in liquid form through an inlet lumen of the cryoprobe at a low positive pressure to the closed distal end in contact with the treatment area, and applying a negative pressure to an outlet lumen of the cryoprobe to remove nitrogen in gaseous form from the treatment area. The applying step may comprise establishing a connection between the outlet lumen and a vacuum source. The low positive pressure for delivery of nitrogen in liquid form may be up to positive 30 psi. The negative pressure may be applied by the vacuum source; the negative pressure may be up to negative 15 psi. The method may further comprise sensing temperature at the closed distal end; the delivery of nitrogen in liquid form and removal of nitrogen in gaseous form may be controlled based on the sensed temperature.

### BRIEF DESCRIPTION OF THE DRAWINGS

In the drawings, like reference characters generally refer to the same parts throughout the different views. Also, the drawings are not necessarily to scale, emphasis instead being placed upon illustrating principles of the present disclosure. The present disclosure, and exemplary embodiments according to the disclosure, are more particularly described in the following description, taken in conjunction with and in reference to the following drawings, in which:

FIG. 1 is a perspective view of a cryosurgery system according to an embodiment of the present disclosure;

FIG. 2 is a perspective view of the interior of a cryosurgery system according to an embodiment of the present disclosure;

FIG. 3A is a schematic showing a cryogen storage, delivery and pressure control apparatus according to an embodiment of the present disclosure;

FIG. 3B is a schematic showing a cryogen storage, delivery and pressure control apparatus according to an embodiment of the present disclosure;

FIG. 4 is an isometric view of a proximal shaft of a cryoprobe according to an embodiment of the present disclosure;

FIG. 5 is a side view of a proximal construction of a cryoprobe according to an embodiment of the present disclosure;

FIG. 6 is a side view of a junction of a larger diameter shaft to a smaller diameter shaft for a proximal construction of a cryoprobe according to an embodiment of the present disclosure;

FIG. 7 is a cross-section view of a bayonet connecter for a cryoprobe according to an embodiment of the present disclosure;

FIG. 8A is a longitudinal cross-section view of a distal construction of a cryoprobe according to an embodiment of the present disclosure;

FIG. 8B is an enlarged view of the distal construction of the cryoprobe of FIG. 8A;

FIG. 9A is a longitudinal cross-section view of a distal construction of a cryoprobe according to an embodiment of the present disclosure;

FIG. 9B is a radial view of the cryoprobe of FIG. 9B looking along the longitudinal axis from the distal end of the cryoprobe;

FIG. 10 is a radial view looking along the longitudinal axis from the distal end of a cryoprobe according to an embodiment of the present disclosure.

### DETAILED DESCRIPTION

Various embodiments according to the present disclosure are described below and with reference to the exemplary configurations of a system and probe, and methods of use thereof, as depicted in the FIGURES.

Exemplary cryosurgery systems, components and parameters thereof, which may implemented in part or whole with the systems, devices and methods of the present invention, include, but are not limited to, the disclosures in U.S. Pat. Nos. 9,301,796 and 9,144,449, entitled “Cryosurgery System”; co-pending U.S. patent application Ser. No. 14/012,320, filed Aug. 28, 2013; and co-pending U.S. patent application Ser. No. 14/809,826, filed Jul. 27, 2015. Each of these patents and applications is incorporated herein by reference in its entirety and for all purposes.

The present invention in its various embodiments is directed to a cryosurgery system having a cryogen delivery apparatus. The cryosurgical system may include a cryogen source configured to provide the cryogen to the cryogen delivery apparatus, a regulation apparatus fluidically coupled to the cryogen source and to the cryogen delivery apparatus, and a controller or console with on-board controls communicatively coupled to the regulation apparatus and configured to control the release of cryogen into the cryogen delivery apparatus. The delivery apparatus may be a catheter, probe or needle configuration that applies a medical-grade liquid nitrogen (or other cryogen) to a treatment area via a small, low pressure, closed end catheter.

In the following description, use of the terms catheter, probe, or needle alone or together is not to be taken as limiting, but rather is exemplary in nature. The disclosure in its various embodiments of a delivery apparatus is meant to encompass the invention broadly in a delivery apparatus, which may include and take the form of one or more of a catheter, probe, needle or other understood term of art. Also, where used herein, “proximal” refers to the relative position on a device that is closer to a physician during use, while “distal” refers to a relative position on the device that is farther from a physician during use.

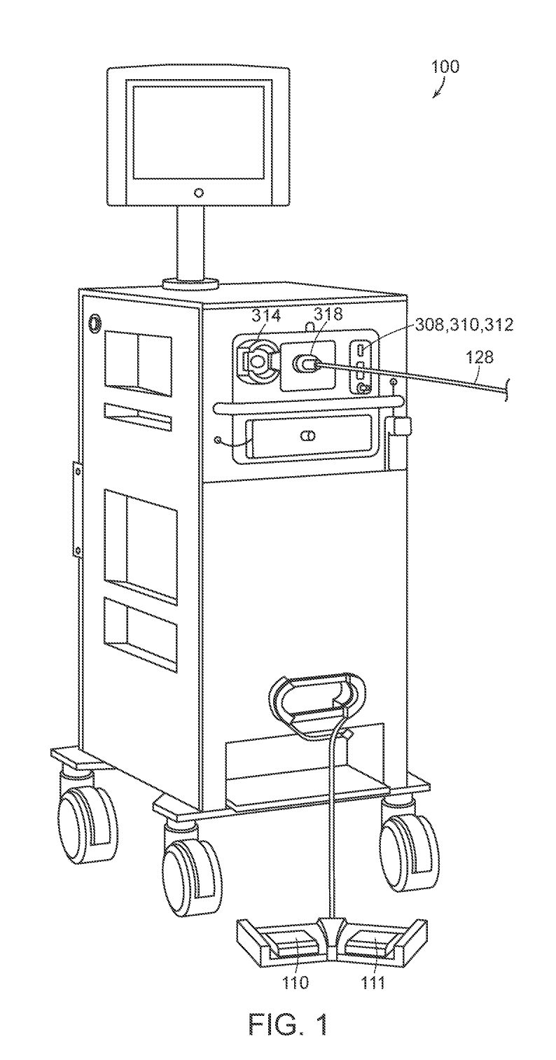

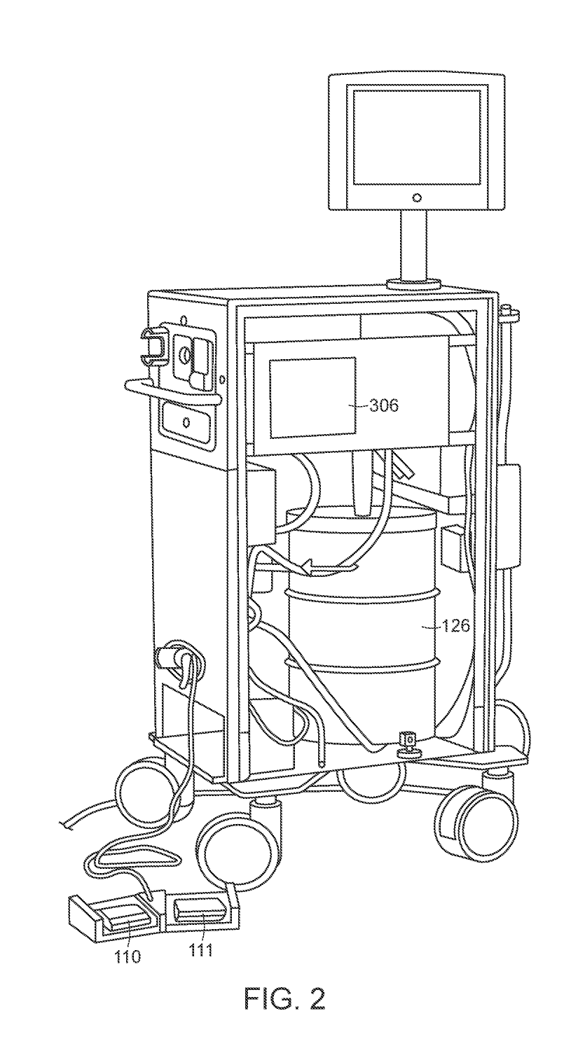

A simplified perspective view of an exemplary cryosurgery system in which embodiments of the present invention may be implemented is illustrated in FIGS. 1 and 2. Cryosurgery system 100 may comprise a pressurized cryogen storage tank 126 to store cryogen under pressure. In the following description, the cryogen stored in tank 126 is liquid nitrogen although cryogen may be other materials as described in detail above. The pressure for the liquefied gas in the tank may range from 5 psi to 50 psi. According to a preferred embodiment, pressure in the tank during storage is 40 psi or less, and pressure in the tank during operation is 35 psi or less. According to a more preferred embodiment, pressure in the tank during storage is 35 psi or less and pressure during operation is 25 psi or less. According to a most preferred embodiment, pressure during operation at normal nitrogen flow is 22±2 psi, and pressure during operation at low nitrogen flow is 14±2 psi. In the context of the output pressure of cryogen from the distal end of the catheter, the term low pressure means 2 psi to 20 psi.

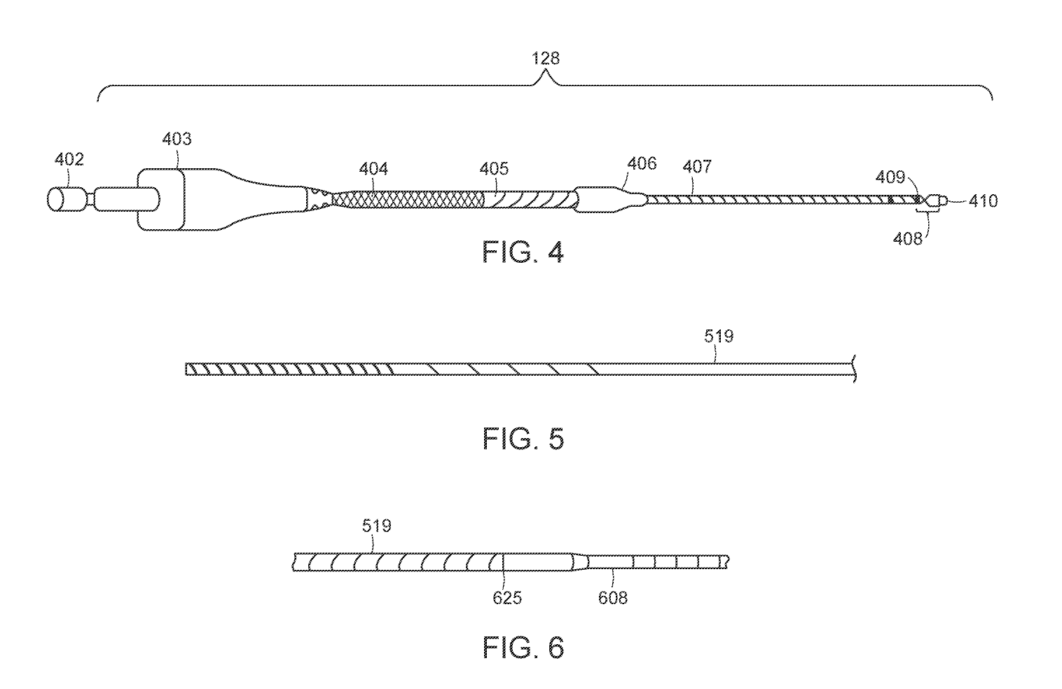

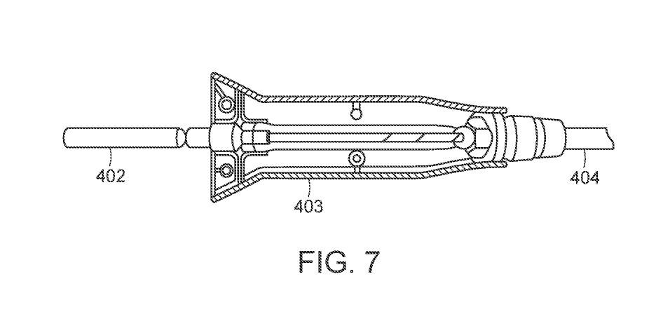

The console depicted in FIG. 1 includes an emergency shut off 314, pressure sensor port 308, temperature sensor port 310 and digital input port 312. An interface 318 is a secure connection point for the delivery apparatus 128 to the console, such as a mating receptacle for a probe connector such as bayonet 402 of probe 128 depicted in FIGS. 4 and 7. The console may include an RFID tag reader 306 to identify each probe 128 as it is used and in the case of a disposable unit, ensure that each probe is only used once per procedure. Foot pedals may be included with system 100 to allow for convenient control of cryogen flow with pedal 110 and suction with pedal 111.

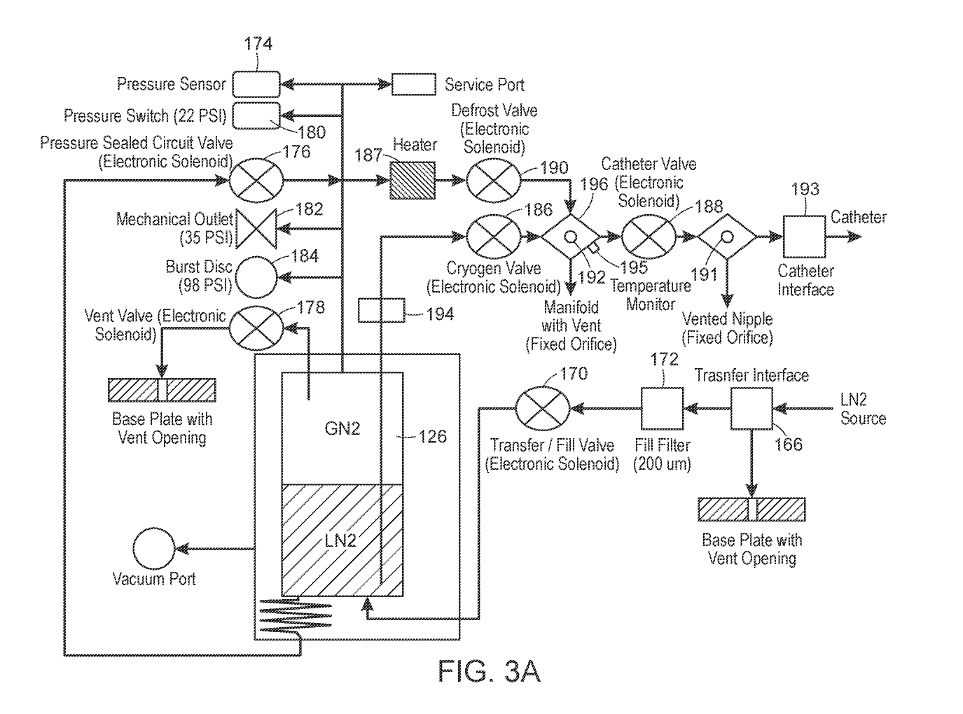

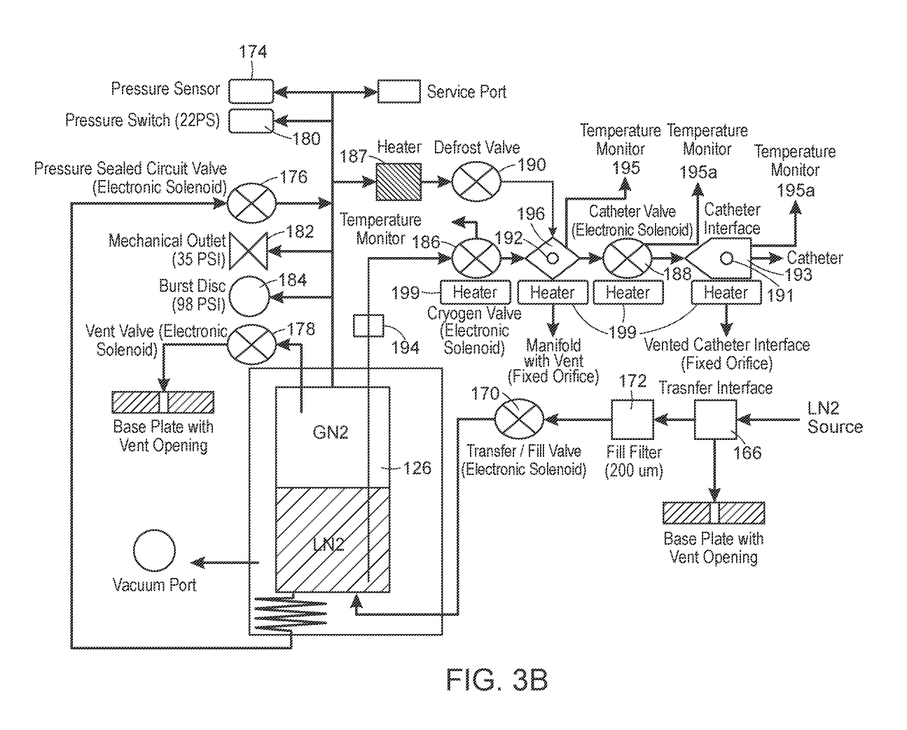

FIGS. 3A and 3B depict flow and control schematics for various embodiments of a console in accordance with the present invention that utilize valves and a pressure sensor 174 to continuously monitor and control the pressure of liquid nitrogen in the tank during use. The console monitors the current pressure of the tank via a pressure sensor 174. The software reads the current pressure from the sensor and adjusts the pressure accordingly. If pressure is too low, the software actuates the pressure build circuit valve 176 to increase the pressure to a specified threshold and then turns off. When the pressure is too high, the software turns on the vent valve 178 until the pressure reaches a specified threshold.

In some cases, system charge pressure is actively controlled by a set of three solenoid valves. A cryogenic solenoid valve connected to the head space is used for rough reduction of tank pressure in cases where tank pressure is significantly above the desired set pressure (>5 psi) or during fill operations when tank pressure must be completely relieved. A set of proportional solenoid valves control the pressure vent and pressure build functions. The proportional solenoid valves are driven by a pulse width modulation (PWM) controller which adjusts its duty cycle based on a control voltage, allowing the valve plunger position to open proportional to the control signal. The control signal is driven by a standard proportional integral derivative (PID) control algorithm executable by a central processor of the system. The PID controller collects data from a precision capacitive pressure sensor and adjusts the valve control signal based on the current pressure deviation with respect to the set point, the current rate of change of pressure, and the pressure history. A PID output control signal determines whether venting or building operations occur. This control scheme advantageously implements precise pressure regulation while allowing software changes to the pressure set point. The PID controller is tuned (inputs P, I, and D) to provide quick response with minimal overshoot or undershoot, while avoiding unstable cycling between vent and build operations.

A mechanical relief valve 182 on the console tank ensures that the tank pressure stays in a safe pressure range. Constant pressure monitoring and adjustment, allows the set point on the mechanical relief valve to be set at a lower pressure, e.g., 35 psi, allowing for a low tank storage pressure. A redundant burst disk 184 provides protection should the mechanical relief valve fail. For optimal safety, both electronic and mechanical pressure valves may be present to regulate the pressure, providing triple redundancy in the event of failure. In addition, a redundant pressure switch 180 may provide accurate tank pressure readings and is checked during the self-test. In an alternate embodiment, the mechanical relief valve 182 may be set at 60 psi, but still allowing to remain a low pressure storage tank.

One or more embodiments of the present invention may utilize a manifold assembly including cryogen valve 186, manifold 196, catheter valve 188, defrost valve 190, fixed orifices 191 and 192, and catheter interface 193 to control liquid nitrogen delivered through the catheter. When the cryogen valve 186 is actuated, liquid nitrogen exits the tank through the lance 194 and proceeds through the cryogen valve 186 to manifold 196 where fixed orifice 192 is present to allow cold expanded gas and liquid cryogen to exit the line and cool down the internal cryogen circuit. During this precool, the catheter valve 188 downstream of the manifold remains closed. A data acquisition board collects data from a thermocouple 195 located on the manifold body. In the precool function, the system software monitors data from the thermocouple 195, and opens the cryogen valve 186 to cool the manifold 196 when its temperature is above the desired set-point. Fixed orifice 191 may be provided on catheter interface 193 to allow venting of cold expanded gas to exit the line during cryogen delivery.

In one embodiment, as represented in FIG. 3B, each of cryogen valve 186, manifold 192, catheter valve 188 and catheter interface 193 may be provided with a temperature thermocouple or sensor 195a and a heater 199 to maintain the cryogen flow path at a constant selected temperature to prevent overcooling of the system resulting from the continuous flow of cryogen through the valves and manifold assembly. According to various embodiments of the invention, each of the heaters may be controlled to maintain the valves, the manifold and the catheter interface at the same temperature or at different temperatures. In one embodiment, the system may be set so that the temperature(s) of the valves, manifold, and catheter interface is/are controlled to be maintained at a temperature greater than −120° C. during cryogenic treatment. The system may be set so that the temperature(s) of the valves, manifold, and catheter interface is/are controlled to be maintained at a temperature of +20° C. during cryogenic treatment. According to another embodiment, each of the valves, manifold, and catheter interface may be controlled and maintained at constant temperatures, but the constant temperatures of each may be different from one or more of the constant temperatures of the others.

A defrost function may be useful for thawing the catheter after cryogen delivery. A defrost circuit directs gaseous nitrogen from the top of the tank through a heater 187 and defrost valve 190 to the catheter 128. When the defrost button on the software screen is pressed, the defrost circuit is activated for a prescribed time (e.g., 30 seconds) but may be stopped earlier at the user's discretion. A low voltage (24 VDC) DC defrost heater delivers 6W minimum of warming/defrost performance but minimizes variation due to line voltage and limits maximum gas temperature, as compared to prior art line voltage (120V) AC heaters.

As liquid nitrogen travels from tank 126 to the proximal end of cryogen delivery catheter 128, the liquid is warmed and starts to boil, resulting in cool gas emerging from the distal end or tip of catheter 128. The amount of boiling in catheter 128 depends on the mass and thermal capacity of catheter 128. Since catheter 128 is of small diameter and mass, the amount of boiling is not great. When the liquid nitrogen undergoes phase change from liquid to gaseous nitrogen, additional pressure is created throughout the length of catheter 128. In an alternate embodiment, the gas boiling inside the catheter may be reduced even greater by the use of insulating materials such as PTFE, FEP, Pebax and others to help reduce its temperature coefficient. The addition of PTFE is especially desirable if done in the inner lumen because its lower coefficient of friction aids in laminar flow of the fluid, thus reducing turbulence and entropy. This reduces gas expansion and allows for good fluid velocity.

The various embodiments of a catheter in accordance with the present disclosure are designed to transport liquid nitrogen (or other cryogen) from a console to a patient treatment site. According to one embodiment, with reference to FIG. 4, a catheter 128 may contain a bayonet 402 and connection housing 403 for attachment to a console at its proximal end, a laser cut hypotube to minimize kinking and breaking, and a polymer layer disposed over the hypotube, thereby sealing the catheter 128, and an insulation layer 404 to protect the user from cold. The hypotube may be spirally cut, imparting radial flexibility while maintaining some axial stiffness and pushablility, and the relative flexibility of the hypotube may be, in some cases, variable along the length of the catheter 128 through the use of a variable-pitch spiral cut. This may be accomplished by varying the separation of the spiral or repeated cut pattern, as well as varying the shape of the pattern itself. For instance, the spiral cut may be characterized by a first, relatively large pitch proximally, and a second, smaller pitch more distally, allowing the distal end, and particularly the tip, to bend about a tighter curve than the most proximal portions of the catheter. The strength and flexibility provided by catheters according to these embodiments may allow a user (e.g., a physician) to retroflex the catheter during a treatment procedure, if needed.

The delivery catheter may be constructed out of hypotubes of different internal diameters mated to each other to make a proximal shaft and a distal shaft, with the distal shaft containing the smaller ID. The proximal and distal shafts may be joined at a connector. The distal shaft may have a reduced ID to be able to fit through a working channel of a scope or trocar. The hypotubes may be laminated with a polymeric heat shrink which seals the laser cut pattern from the liquid intended to flow inside the catheter.

The polymer layer may be any suitable flexible polymer that is substantially gas impermeable (for example fluorinated ethylene propylene or urethane), and may be disposed over the hypotube in the form of one or more extrusion layers attached by means of heat shrinking, or by means of dip coating, melt coating or spray coating. The catheter package may contain an RFID tag that the user scans prior to use to prevent reuse and track disposable information. An alternative construction locates the RFID tag on the connector area adjacent to the bayonet, such that a RFID tag may be scanned by the system, such as by RFID reader 306, when the catheter is connected to the system.

The delivery catheter in other embodiments may be constructed of one or more layers of flexible polyimide, surrounded by a stainless steel braid, which is in turn coated with an outer layer of Pebax. Extrusion of Pebax over the stainless steel braid may allow the Pebax to wick through the pitch of the steel braid, helping to prevent kinking, breaking, or delamination during retroflex of the catheter. The Pebax may also provide a desirable balance between hardness, which is important for smooth sliding of the catheter and general toughness, and softness, which is important for some degree of tackiness which allows the user to feel the movement of the catheter during insertion. The pitch of the stainless steel braid can be configured to be fine enough to afford the required strength, but still allow the Pebax to wick through.

Referring again to FIG. 4, an embodiment of a cryogenic catheter 128 is depicted, which includes bayonet connection 402, catheter connection housing 403, insulation 404, laser cut hypotube with FEP or Pebax heat shrink wrap 405, nozzle connection of diminishing inner diameter 406, second smaller ID laser cut hypotube 407 with FEP or Pebax heatshrink wrap, catheter/needle head 408, marking band 409, and closed distal end 410. FIG. 7 depicts the insulator 404 and an exemplary cross-section of connection housing 403 with bayonet 402 at the proximal end of catheter assembly 128 for attachment to a cryogen source.

FIG. 5 shows a hypotube 519 that may be used for the construction of the proximal end of the catheter shaft 405. In various embodiments, it may have a length of approximately 45 inches, but can vary from 10 inches to 100 inches in length. The internal diameter of the tube 519 may be approximately 0.104 inches (3.56 mm), but can vary from 0.031 inches to 0.197 inches (0.8 mm to 5 mm), preferably from 0.039 inches to 0.157 inches (1 mm to 4 mm). The hypotube 519 may be, as shown, laser cut as a spiral, but other variable cuts can be present to provide desired flexibility/rigidity along the length of the tube.

FIG. 6 shows a transition 625 of a larger diameter hypotube shaft 519 to a smaller diameter laser cut hypotube shaft 608. The transition is so that a smaller diameter may be inserted for example into the working channel of a scope or trocar. In addition, the transition from large diameter to small diameter may act as a mixing point for dual phase flow gas and liquid to interact along the path of the catheter shaft and allow for the gas to once again attain the velocity of the liquid as the dual phase flow travels down the shaft. This is understood by those skilled in that art as a “nozzling” transition. Control of cryogen suited to desired treatment applications and parameters may be achieved in accordance with the present disclosure through a “nozzle” flow created by tailoring, for example, shafts of a certain length, diameter size and number of transitions. Transitions may occur between two hypotubes, two polymeric shafts or between a coil and hypotube or coil and polymeric shaft.

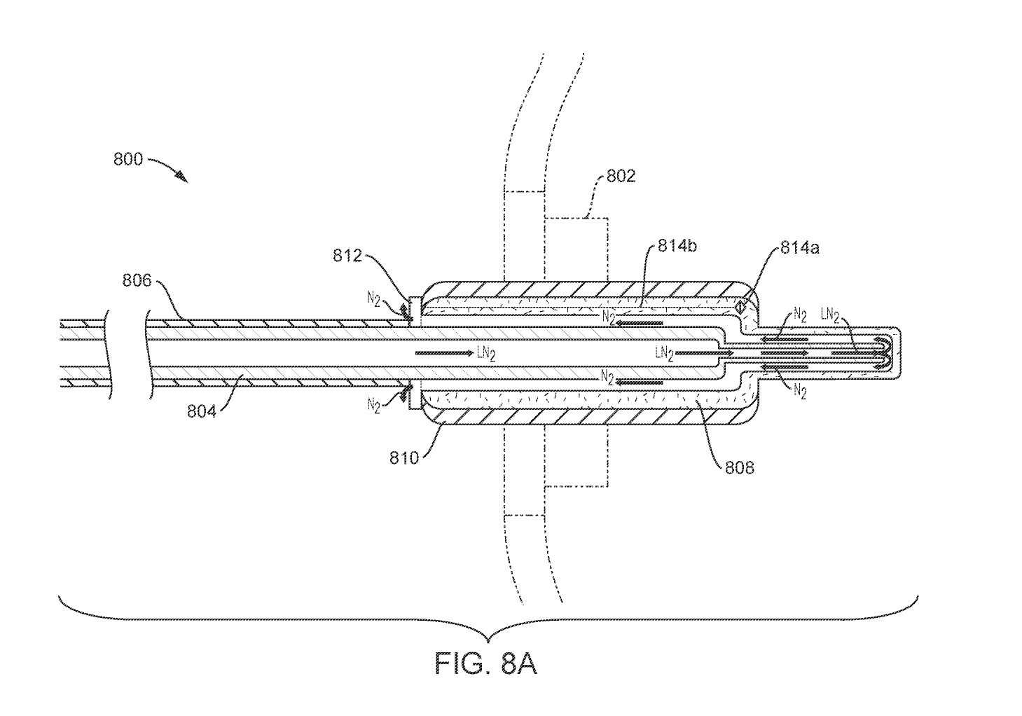

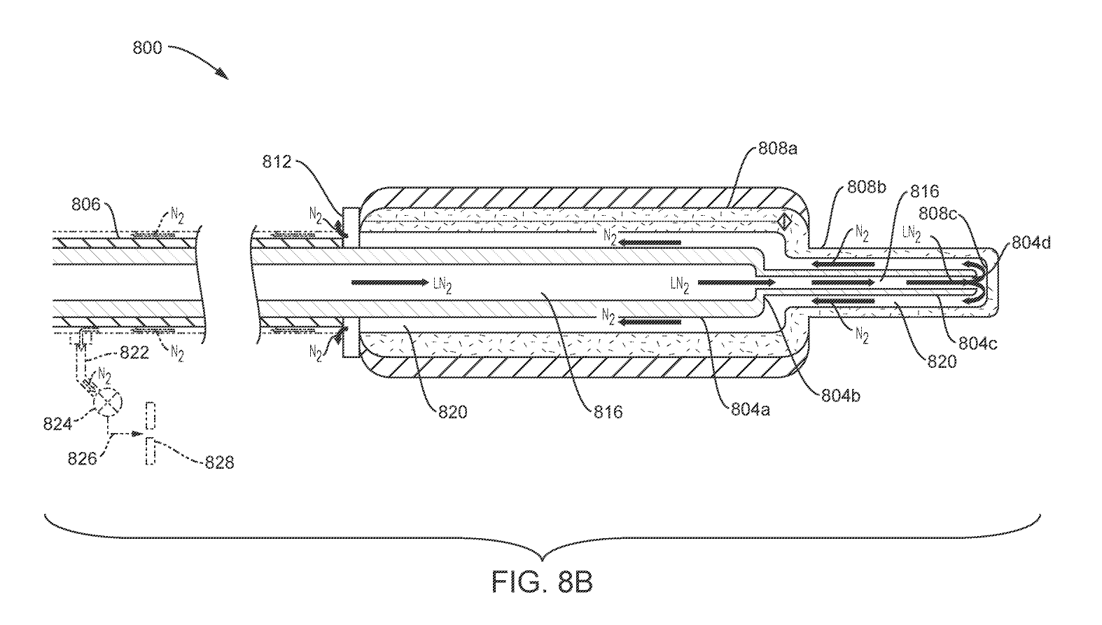

Various configurations in accordance with the present disclosure for the distal end of a catheter, such delivery apparatus 128 of FIG. 4, with catheter head 408 and closed distal end 410, are described with reference to FIGS. 8-10. The exemplary embodiments described, including the dimensions, materials, flow and pressure parameters, are in the context of liquid nitrogen delivery to a treatment site under direct laparoscopic visualization with the cryoprobe inserted through a conventional trocar set-up (e.g., trocar 802 of FIG. 8A). Variations on one or more of these parameters, including for example use of a different cryogen source or sizing of a catheter for insertion through the working channel of an endoscope, may be readily determined by one skilled in the art and are within the intended scope of the present disclosure.

FIGS. 8A and 8B depict a single needle embodiment of a cryoprobe head 800, in accordance with the present invention, at a distal end of the delivery apparatus 128. Liquid nitrogen flows along inlet path 816 into inner jacket 804. Inner jacket is configured as a tube with larger diameter portion 804a transitioning at the inner jacket shoulder 804b to smaller diameter portion 804c, and terminating at inlet opening 804d. The inner jacket is surrounded co-axially by outer jacket 808, which includes contact face 808c across from inlet opening 804d, and smaller diameter portion 808b transitioning to larger diameter portion 808a. The relative inner diameters of the outer jacket and inner jacket are maintained such that a channel forms between the two and defines outlet flow path 820, as liquid nitrogen exits the inner jacket 804 at opening 804d and travels along the channel to the proximal end of outer jacket 808. A diffuser 812 at the outlet of outer jacket 808 ensures that any residual liquid nitrogen is converted to gaseous nitrogen before it exits probe head 800. Inner jacket 804 and outer jacket 808 include, respectively, insulation 806, 810 around portions of the exterior of the jackets where an insulating effect is desirable and exposure to the user and patient is not desired. Gaseous nitrogen exits to the atmosphere directly from diffuser 812, as shown, or may follow a path directed by an optional vacuum source before venting.

An alternative embodiment according to the present invention that utilizes a vacuum source is depicted in FIG. 8B. Instead of exiting directly to atmosphere at the proximal side of diffuser 812, the gaseous nitrogen continues along an extension of outer jacket 808 that is in fluid communication with pump 824. A fitting on the extension transitions to pump inlet 822 leading to the pump. Pump outlet 826 carries gaseous nitrogen from the pump to vent 828 where the gaseous nitrogen is vented to the atmosphere. Use of pump 824, or other vacuum source, allows a negative pressure to be applied to the outlet flow path 820 of gaseous nitrogen. A negative pressure (or pressure below atmospheric pressure) may be applied from 0 up to 760 Torr below atmosphere, which is equivalent 0-14.5 psi of vacuum. The resulting higher pressure differential between the liquid nitrogen entering the delivery apparatus through the inner jacket (e.g., 14.5 psi positive pressure) and the gaseous nitrogen exiting the delivery apparatus through the outer jacket (e.g., 14.5 psi negative pressure), adds capability within the system to drive more liquid nitrogen through the catheter per unit time with concurrent enhancement in targeted tissue cooling, while still maintaining a low pressure liquid nitrogen inlet system.

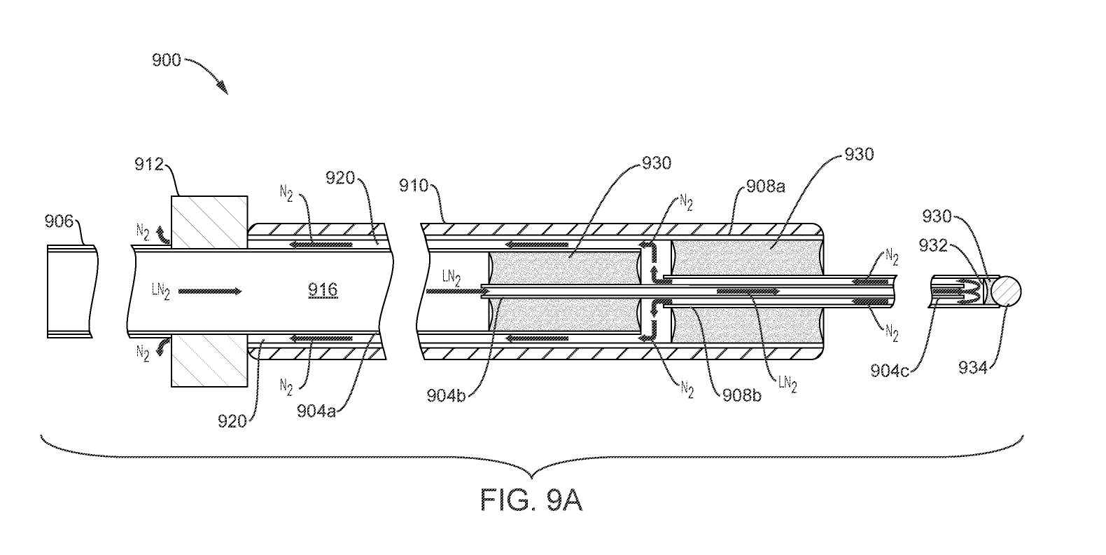

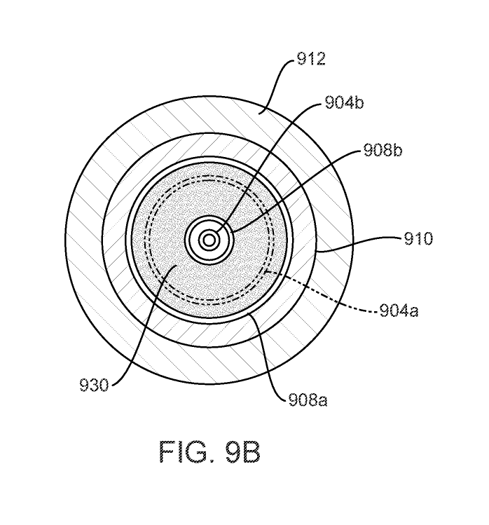

FIGS. 9A and 9B depict an alternate single needle embodiment of a cryoprobe head 900, in accordance with the present invention, at a distal end of the delivery apparatus 128. Liquid nitrogen flows along inlet path 916 into inner jacket 904. Inner jacket is configured as two pieces of tubing: the first piece, larger diameter portion 904a, transitions to the second piece, smaller diameter portion 904b, which terminates at inlet opening 904c. Smaller diameter portion 904b extends through and is secured within the interior of larger diameter portion 904a by an insulating adhesive material 930 forming a plug at the distal end of larger diameter portion 904a. The inner jacket is surrounded co-axially by outer jacket 908. Outer jacket is also configured as two pieces of tubing: the first piece, larger diameter portion 908a, transitions to the second piece, smaller diameter portion 908b, which terminates at backstop 932, adhesive material 930 and contact face 934, across from the inlet opening 904c. In the embodiment depicted, contract face 934 is in the form of a ball tip that provides an atraumatic contact surface for the target tissue, but other shapes and forms may be suitable. Smaller diameter portion 908b extends through and is secured within the interior of larger diameter portion 908a by insulating adhesive material 930 forming a plug at the distal end of larger diameter portion 908a.

The relative inner diameters of the outer jacket and inner jacket are maintained such that a channel is formed between the two that defines an outlet flow path 920 as liquid nitrogen exits the inner jacket 904 at opening 904c and travels along the channel to the proximal end of outer jacket 908. A diffuser 912 at the outlet of outer jacket 908 ensures that any residual liquid nitrogen is converted to gaseous nitrogen before it exits probe head 900. Inner jacket 904 and outer jacket 908 include, respectively, insulation 906, 910 around portions of the exterior of the jackets where an insulating effect is desirable and exposure to the user and patient is not desired. Gaseous nitrogen exits to the atmosphere directly from diffuser 912, as shown, or may follow a path directed by an optional vacuum source before venting, for example, similar to the extension of the outer jacket and pump arrangement depicted in FIG. 8B. FIG. 9B is a view of the catheter head 900 from the distal tip showing the relative diameters of the inner and outer jacket as they each transition from a larger diameter to smaller profile terminating at the distal needle ball tip end 934.

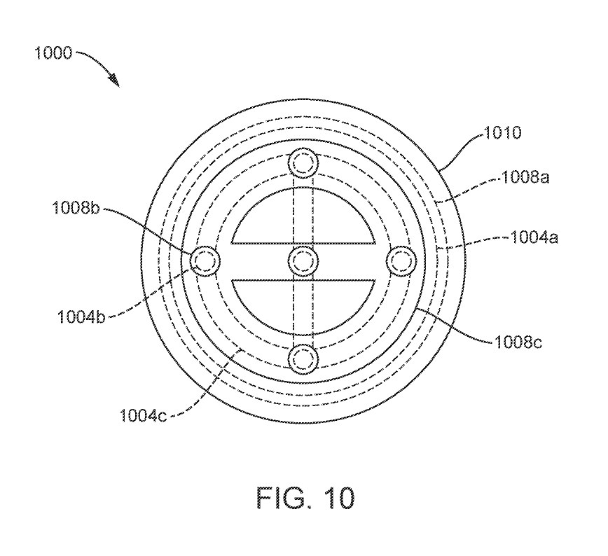

The various needle/probe embodiments in accordance with the present invention may be configured as a single needle, such as described with reference to FIGS. 8 and 9, or the distal end of the catheter head may be configured with multiple needles at the tip. FIG. 10 depicts an exemplary multiple needle embodiment viewed from the distal tip of catheter head 1000. Inner and outer jackets 1004, 1008 may have larger diameter portions 1004a, 1008a that transition to smaller diameter portions 1004b, 1008b, similar to the arrangements described with respect to FIGS. 8 and 9. However, the respective transitions of catheter head 1000 take the form of concentric manifolds 1004c, 1008c, with the manifold of the inner jacket 1004c within the manifold of the outer jacket 1008c, terminating at the inlet opening of the five needle tips 1004b. The liquid nitrogen exiting the inlet openings returns as gaseous nitrogen along the path of the smaller diameter portions 1008b at each of the five needle tips, along manifold 1008c, and then along the larger diameter portion 1008a to the outlet and diffuser 1010 at the proximal end of catheter head 1000.

In various embodiments according to the present disclosure, the probe head may include a temperature sensor. FIGS. 8A and 8B, for example, depict a thermocouple sensor 814a and wire 814b. This may be achieved by laying at least two wires longitudinally or in a coil pattern prior to an outer layer of insulation, such as insulation 810, being applied to the exterior of catheter head 800. Wire that are thermocouple wires, for example, constantin and copper, may be terminated into a thermocouple. Alternatively, a cryogenic thermistor may be attached to the distal end of the catheter head 800. Such a thermistor may be encapsulated, for example, with conductive epoxy and a polymeric sleeve. The thermocouple, thermistor or another sensor may be used to monitor and report temperatures, including as part of a control feedback loop for control of cryogen flow, both at the tip of the catheter head as well as the treatment area. In a thermocouple wire construction, the wires may be integrated outside of or within the shaft construction proximal to the catheter head 800. The thermocouple wires may be connected to a console such as the console of system 100 in FIGS. 1-2, via contacts 310 within the console housing.

Various shapes, number and configuration of closed-tip needles are contemplated within the scope of the present disclosure. The needle tips may have blunt contact surfaces, such as depicted and described with respect to FIGS. 8-10, or the tips may be sharp in order that the needle tips may be penetrated into target tissue during cryotherapy.

Exemplary dimensions for the inner and outer jackets 804, 808 of catheter head 800 include: Inner jacket: the larger diameter portion 804a may have an ID of 0.104″ (2.64 mm) and an OD of 0.112″ (2.84 mm); the smaller diameter portion 804c may have an ID of 0.010″ (0.26 mm) and an OD of 0.018″ (0.46 mm or 26 gauge); Outer jacket: the larger diameter portion 808a may have an ID of 0.140″ (3.56 mm) and an OD of 0.150″ (3.81 mm); the smaller diameter portion 808b may an ID of 0.027″ (0.80 mm) and an OD of 0.042″ (1.07 mm or 19 gauge). The overall OD of the catheter head 800 at the larger diameter portion including the insulation 810 may be 0.18″ (4.57 mm).

Exemplary dimensions for the inner and outer jackets 904, 908 of catheter head 900 include: Inner jacket: the larger diameter portion 904a may have an ID of 0.104″ (2.64 mm) and an OD of 0.112″ (2.84 mm); the smaller diameter portion 904b may have an ID of 0.010″ (0.26 mm) and an OD of 0.018″ (0.46 mm or 26 gauge); Outer jacket: the larger diameter portion 908a may have an ID of 0.135″ (3.43 mm) and an OD of 0.148″ (3.76 mm); the smaller diameter portion 908b may an ID of 0.035″ (0.89 mm) and an OD of 0.042″ (1.07 mm or 19 gauge).

Exemplary material for the inner and outer jackets include surgical grade stainless steel or nitinol hypotubes that are, for example, laser cut to desired configurations. Ball tip 934 may be surgical grade stainless steel. Exemplary material for insulations 806, 810, 906, 910 include shrink wrap polyimide, FEP, PTFE, and PEBAX, among others. Material 930 may be an epoxy adhesive. Dimensions and materials for the jackets, insulation and needle tips may be varied in accordance with the present disclosure, and choices for an intended purpose may be readily determined by one skilled in the art in order to optimize a particular configuration or treatment protocol.

Methods according to various embodiments of the present invention involve the use of contact cryotherapy, which when the treatment site is internal to the body, includes visual guidance of a laparoscope or endoscope (in its broadest interpretation, endoscope is intended to include all forms of scopes that are configured for access through a natural opening in the body, as compared to the percutaneous access of a laparoscope, including but not limited to, gastroscope, ENT scope, colonoscope, ureteroscope, cystoscope, hysteroscope, bronchoscope). While described with respect to therapy at sites internal to the body, the systems and devices disclosed are applicable as well to contract cryotherapy external to the body, such as dermatological treatment of lesions, tumors, etc.

In either of the internal or external approaches, a physician or other user, in accordance with the various embodiments of the invention, attaches the proximal end of a catheter to a source of cryogen, such as by mating bayonet 402 of the catheter connection housing 403 to the catheter interface 318, and liquid nitrogen source 126, of the console of system 100 in FIGS. 1-2. Various sensor inputs may be attached as well, for example thermocouple 814a via wires 814b. On-board controls may be available for the purpose of, as examples, pre-cooling the catheter, calibrating the system, monitoring pressure in the source tank, monitoring temperature at the catheter distal end and setting the parameters for the cryogen delivery treatment protocol.

Feedback loop and software controls may be utilized that meter the cryogen delivery based on feedback that is received from the system, for example, dosing parameters calculated based on the maintenance of a certain level of liquid nitrogen temperatures at the treatment area for predetermined time periods. An example of a suitable cryogen source and console set-up and controls for low pressure delivery of liquid nitrogen is the TruFreeeze® system, available from CSA Medical, Inc.; provided, however, catheters configured according to the present disclosure for interface with an alternative source of low pressure cryogen would be suitable as well.

Once the proximal end of the delivery apparatus is attached to a cryogen source, and system set-up is complete, the apparatus may be inserted into the body of the patient proximate the treatment site. Insertion may be achieved through a trocar independent of the working channel of laparoscope, such as shown in FIG. 8A, in which case visual guidance will be provided independently through the same port or a different port. Alternatively, the catheter is inserted through the working channel of a scope, which could be either a laparoscope or endoscope, depending on the configuration of the catheter. In embodiments utilizing a vacuum source, a pump or other source of suction is attached to the gaseous nitrogen outlet of the catheter outer jacket, for example, pump inlet 822 and pump 824 attached to jacket 808a of catheter head 800 in FIG. 8B.

Cryogen delivery is started and maintained for the duration of the procedure with flow, and optionally suction, being operated via manual or automatic controls, such as, respectively, foot pedals 110, 111, alone or in conjunction with electronic feedback loop control tied to temperature monitoring. Cryogen, e.g., liquid nitrogen, flows at low pressure (e.g., 14.5 psi) through the catheter shaft into the distal tip of the catheter head. At the transition point, the liquid nitrogen passes into a reduced diameter section of tubing, such as the transition at shoulder 804b from the larger ID (e.g., 2.64 mm) portion 804a of inner jacket 804 to the smaller ID (e.g., 26 gauge, 0.46 mm) needle portion 804c. Upon exiting the smaller diameter tubing, the cryogen impacts upon the contact face of the outer jacket, such as the flow of liquid nitrogen (designated as 816 in FIGS. 8A and 8B) out of the inlet opening 804d impacting contact face 808c of smaller diameter needle portion 808b of outer jacket 808. In the embodiment depicted in FIG. 8, liquid nitrogen converts to gaseous nitrogen and flows back along path 820 toward the proximal end of the catheter head and exits the outlet of larger diameter portion 808a of outer jacket 808 through diffuser 812. At the proximal side of the diffuser the nitrogen exits the catheter to the atmosphere or, if an optional vacuum source is used, the nitrogen gas is pulled along larger diameter portion 808a through pump inlet 822 and exits the pump to vent 828 through pump outlet 826.

Embodiments of the methods, devices and system, as described above, and otherwise in accordance with the present invention, result in greater throughput of liquid nitrogen, e.g., more liquid nitrogen at the contact face in a given amount of time, resulting in faster freeze times, particularly when a vacuum source is applied versus conventional closed systems. Faster freeze times are thought to enhance cell death and treatment efficacy since the water in the cells is frozen before the cell dehydrates, expanding within the cells and causing cell death when the ice thaws.

Liquid nitrogen temperatures (e.g., 77 Kelvin) are able to be achieved with cryoprobes according to the present invention while maintaining low pressure input of liquid nitrogen (such as 20 psi) on the inlet side. The lower inlet pressure allows for lower profile needle dimensions, while still maintaining the throughput of liquid nitrogen necessary to achieve the necessary treatment temperatures.

While the examples presented above may be focused on treatment of particular anatomy, the systems, methods, and principles illustrated thereby, alone or in a system or kit or as part of a method or procedure, including with other accessories, will be understood by those skilled in the art to be applicable to cryotherapy of other systems and conditions within cavities, lumens, tracts, vessels and organs of the body, in which delivery of cryogen to a site, including the esophagus, peritoneal, abdominal, bronchial or thoracic cavities, vasculature, gastrointestinal or urinary tract, uterus, bladder, lung, liver, stomach, duodenum, small intestine, large intestine, rectum, fallopian tube, etc., is desired.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example.

Certain embodiments of the present invention have been described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the scope of the present disclosure is not to be limited by the preceding illustrative description, but instead is defined by the following claims.

1. A cryosurgical system comprising:
a cryogen source;
a vacuum source; and
a cryogen delivery apparatus configured to (i) connect to the vacuum source and the cryogen source, (ii) deliver cryogen in liquid form from the cryogen source through the apparatus at a low positive pressure to a treatment area, and (iii) remove cryogen in gaseous form from the treatment area through the apparatus at a negative pressure produced by the vacuum source.

2. The system according to claim 1, wherein the cryogen delivery apparatus is a catheter having a closed distal end.

3. The system according to claim 2, wherein the closed distal end has one or more blunt tips to contact a surface of the treatment area.

4. The system according to claim 2, wherein the closed distal end has one or more needle tips to penetrate a surface of the treatment area.

5. The system according to claim 1, wherein the low positive pressure is up to positive 20 psi.

6. The system according to claim 1, wherein the negative pressure is up to negative 15 psi.

7. The system according to claim 1, wherein the cryogen source is nitrogen in liquid form.

8. The system according to claim 2, further comprising a console having on-board controls and a temperature sensor in electrical communication with the controls and coupled to the closed distal end of the catheter in a feedback loop arrangement that allows for control of a rate of cryogen delivered and removed by the system based on temperature measured by the sensor.

9. An apparatus for delivery of cryogen to a treatment area within a body, comprising:
a proximal attachment end for connection to a cryogen source;
a closed distal end having a head with one or more low profile tips to contact the treatment area; and
a shaft comprising a first inlet lumen and a second outlet lumen, the first inlet lumen extending from the proximal end to deliver cryogen in liquid form to the tips under low positive pressure, the second outlet lumen extending from the one or more tips to vent cryogen in gaseous form from the treatment area to atmosphere outside the body.

10. An apparatus according to claim 9, wherein the one or more low profile tips have a blunt face to contact a surface of the treatment area.

11. An apparatus according to claim 9, wherein the one or more low profile tips are sufficiently sharp to penetrate a surface of the treatment area.

12. An apparatus according to claim 9, wherein an outer diameter of the first inlet lumen within the one or more low profile tips is no more than 26 gauge.

13. An apparatus according to claim 9, wherein an outer diameter of the second outlet lumen within the one or more low profile tips is no more than 19 gauge.

14. An apparatus according to claim 9, wherein the cryogen source is nitrogen in liquid form and the low positive pressure is up to positive 30 psi.

15. An apparatus according to claim 9, wherein the second outlet lumen has a connection for a vacuum source and is configured to vent the cryogen in gaseous form with a vacuum pressure up to negative 15 psi.

16. An apparatus according to claim 9, wherein the first inlet lumen is arranged co-axially within the second outlet lumen leaving a channel therebetween in fluid communication with the first inlet lumen that defines a flow path to vent the cryogen in gaseous form from the treatment area.

17. A method to deliver cryotherapy to a treatment area, comprising:
positioning a closed distal end of a cryoprobe in contact with the treatment area;
delivering nitrogen in liquid form through an inlet lumen of the cryoprobe at a low positive pressure to the closed distal end in contact with the treatment area; and
applying a negative pressure to an outlet lumen of the cryoprobe to remove nitrogen in gaseous form from the treatment area.

18. The method according to claim 17, wherein the applying step comprises establishing a connection between the outlet lumen and a vacuum source.

19. The method according to claim 18, where the low positive pressure is up to positive 30 psi and the negative pressure is applied by the vacuum source up to negative 15 psi.

20. The method according to claim 17, further comprising sensing temperature at the closed distal end and controlling the delivery of nitrogen in liquid form and removal of nitrogen in gaseous form based on the sensed temperature.
